(19) United States
(12) Patent Application Publication
(10) Pub. No.: US 2021/0299034 A1
(43) Pub. Date: Sep. 30, 2021

(54) A Topical Composition

(71) Applicant: Conopco, Inc., d/b/a UNILEVER, Englewood Cliffs, NJ (US)

(72) Inventors: Aparna DAMLE, Bangalore (IN); Anita DAMODARAN, Bangalore (IN); Annu KUMARI, Keonihar (IN); Deepak Ramachandra MHASAVADE, Karntataka (IN); Uma SANTHANAM, Tenafly, NJ (US)

(73) Assignee: Conopco, Inc., d/b/a UNILEVER, Englewood Cliffs, NJ (US)

(21) Appl. No.: 17/265,177

(22) PCT Filed: Jul. 18, 2019
(86) PCT No.: PCT/EP2019/069318

§ 371 Date: Feb. 1, 2021

(30) Foreign Application Priority Data
Aug. 6, 2018 (EP) 18187489.2

(51) Int. Cl. A61K 8/9789; A61Q 17/04; A61Q 19/02; A61K 8/368

(52) U.S. Cl. CPC A61K 8/9789; A61Q 17/04; A61K 2800/5422; A61K 8/368; A61Q 19/02

(57) ABSTRACT

The present invention relates to a topical composition. Particularly, the present invention relates to a topical composition that comprises degallated tea extract that comprises less than 0.2% gallic acid on dry weight basis and provides improved skin lightening.

### FIELD OF THE INVENTION

The present invention relates to a topical composition. Particularly, the present invention relates to a topical composition that provides improved skin lightening.

### BACKGROUND OF THE INVENTION

People often try to take care of themselves with a desire of enjoying a healthy lifestyle. One of the ways people do so is through taking care of their body surfaces e.g. skin, scalp including hair and oral cavity. Some of the benefits people tend to have desire for include healthy and infection-free skin, even skin tone, adequate moisturization, anti-aging, skin lightening and protection from ultraviolet radiation contained in the sunlight.

Skin, being the outermost layer of the human body, is often exposed to factors e.g. pollution, dust, dirt, heat, humidity and ultraviolet radiation contained in sunlight. While the skin protects the body from these factors, at the same time, it is prone to suffer unwanted effects that may be caused due to one or more of said factors. For example, overexposure to ultraviolet radiation contained in sunlight is said to be associated with conditions like tanning, blotchy skin, hyperpigmentation, freckles, melasma which may in turn lead to e.g. uneven skin tones which is not preferred by some consumers.

Therefore, as much as possible, people tend to avoid getting exposed to at least some of said factors. However, in many instances, avoiding exposure to factors e.g. sunlight, is difficult and at times, impossible. As a result, people keep getting exposed to one or more of said factors.

It is for reasons like these, people tend to make use of various topical compositions e.g. sunscreen composition, skin lightening composition, moisturizing composition and anti-aging composition. Such topical compositions offer at least some benefit to the skin e.g. protection against unwanted effects caused by excessive exposure to UV radiations contained in sunlight, skin lightening and moisturizing benefit.

Many of these benefits may be delivered by one or more active ingredients (actives) contained in a topical composition. For example, U.S. Pat. No 4,096,240 (Unilever) discloses that niacinamide comprised in a topical composition provides skin lightening. An active like vitamin C is reported to deliver several benefits that include its use as an antioxidant, an anti-inflammatory agent and depigmentation agent (Telang 2013, Indian Dermatol Online J 4(2): 143-146). Hydroquinone and picolinamide are reported to be effective in treating melasma (Mohammad et al, 2014, Biosciences Biotechnology Research Asia 11(2): 1047-1050).

Additionally, extracts obtained from plant of genus Camellia e.g. Camellia sinensis (C. sinensis) are also used as actives. C. sinensis var. sinensis and C. sinensis var. assamica are widely known varieties of C. sinensis. For example, WO13060710 (Unilever) discloses green tea extract and black tea extract of C. sinensis to be associated with skin lightening. Gallic acid contained in tea extracts is said to be associated with regulation of skin photoaging in UVB exposed fibroblast and hairless mice (Hwang et al., 2014, Phytotherapy Research, 28, 1778-1788). Gallic acid-loaded gel formulation is said to combat skin oxidative stress (Monterio e silva et al; Polymers 2017, 9, 391).

U.S. Pat. No. 6,551,602 (Unilever) discloses a skin care composition containing conjugated linoleic acid and a phenolic compound e.g. epigallocatechin gallate (EGCG), in a dermatologically acceptable base. In addition to anti-aging benefit, the compositions disclosed therein are also said to deliver benefits like soothing sensitive and/or irritated skin, controlling oil/sebum secretion and for lightening the skin.

Despite there exist various actives that provide some or the other benefits as outlined above, people tend to look for one or more actives that deliver improved benefits, e.g. improved skin lightening.

Need therefore exists to develop a topical composition that provides improved skin lightening.

It has now been found that a topical composition comprising degallated tea extract wherein the extract comprises less than 0.2% gallic acid on dry weight basis, provides improved skin lightening.

### SUMMARY OF THE INVENTION

In a first aspect, the present invention relates to a topical composition comprising from 0.01 to 10 wt % degallated tea extract wherein the extract comprises less than 0.2% gallic acid on dry weight basis.

In a second aspect, the present invention relates to a method of providing skin lightening wherein the method comprises the steps of:
i. applying on the skin the composition according to the first aspect; and
ii. optionally, rinsing with water.

In a third aspect, the present invention relates to use of the composition according to the first aspect for skin lightening.

### DETAILED DESCRIPTION OF THE INVENTION

The word “comprising” is intended to mean “including” but not necessarily. “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated. Unless specified otherwise, amounts as used herein are expressed in percentage by weight based on total weight of the composition and is abbreviated as “wt %”. The use of any and all examples or exemplary language e.g. “such as” provided herein is intended merely to better illuminate the invention and does not in any way limit the scope of the invention otherwise claimed.

In a first aspect, the present invention relates to a topical composition comprising from 0.01 to 10 wt % degallated tea extract wherein the extract comprises less than 0.2% gallic acid on dry weight basis.

### Degallated Tea Extract

The composition according to the present invention (the composition) comprises degallated tea extract wherein the extract comprises less than 0.2% gallic acid on dry weight basis (abbreviated and hereinafter referred to as ‘DGTE’).

It will be understood that DGTE means a tea extract that is degallated to an extent that it comprises less than 0.2% gallic acid, on dry weight basis.

Accordingly, DGTE comprises less than 0.2%, preferably less than 0.1%, more preferably less than 0.01%, even more preferably less than 0.001% and further more preferably less than 0.0001% gallic acid on dry weight basis. Most preferably, the DGTE comprises 0% gallic acid, on dry weight basis.

Preferably, the DGTE further comprises from 5 to 25%, more preferably from 7 to 22%, even more preferably from 9 to 20%, further more preferably from 11 to 18% caffeine, on dry weight basis.

Preferably, the DGTE is obtained using tea material obtained from C. sinensis.

Examples of tea material obtained from C. sinensis include fresh or frozen leaves of C. sinensis var. sinensis, C. sinensis var. assamica, instant black tea, instant green tea, instant oolong tea, instant white tea, instant puerh tea, commercially available extracts of black tea, commercially available extracts of green tea, juice obtained by squeezing fresh leaves of C. sinensis var. sinensis, C. sinensis var. assamica and mixtures thereof.

Preferably, the tea material is selected from instant black tea, instant green tea, commercially available extracts of green tea, commercially available extracts of black tea, juice obtained from leaves of C. sinensis var. sinensis and C. sinensis var. assamica and mixtures thereof. More preferably, the tea material is selected from commercially available extracts of green tea, commercially available extracts of black tea and mixtures thereof.

Preferably, the DGTE of the invention is prepared using the following steps:
a) subjecting a tea material obtained from C. sinensis to an enzymatic hydrolysis,
b) separating gallic acid from the enzyme hydrolyzed tea material of step a); and
c) drying the remaining tea material obtained in step b).

### Step a)

In step a) a tea material obtained from C. sinensis as described above, is subjected to an enzymatic hydrolysis. Examples of enzymes that may be used in enzymatic hydrolysis of the tea material include ‘tannin acyl hydrolase’ (tannase; EC number 3.1.1.20). Tannase may be used to carry out enzymatic hydrolysis of gallated species, i.e. epigallocatechin gallate (EGCG), epicatechin gallate (ECG), that is present in the tea material selected in step a). Upon treatment with Tannase, the gallated species get converted to epigallocatechin (EGO) and epicatechin (EC) and gallic acid is generated.

The enzymatic hydrolysis using tannase is preferably carried out at temperature ranging from 20 to 55° C., more preferably from 25 to 50° C., even more preferably from 30 to 45° C. and most preferably from 35 to 40° C. The enzymatic hydrolysis may preferably be carried out for a duration ranging from 30 to 90 min, more preferably from 45 min to 75 min, even more preferably from 50 min to 60 min. The step a) is preferably carried out at pH ranging from 3 to 7, more preferably from 4 to 6 and most preferably at pH 5.5.

The enzyme hydrolyzed tea material obtained at the end of step a) may be stored at temperatures ranging from 0 to 4° C. until used further in step b).

### Step b)

In step b), the enzyme hydrolyzed tea material obtained after step a) is subjected to a separation technique to separate gallic acid from the enzyme hydrolyzed tea material of step a).

Examples of separation techniques that may be used in step b) include adsorption chromatography, anion exchange chromatography, liquid-liquid separation and combinations thereof.

Preferably, the separation technique that may be used in step b), is adsorption chromatography where, the enzyme hydrolyzed tea material obtained in step a) is passed through an adsorption resin column allowing gallic acid generated in step a) to pass through the column retaining degallated species on to the column. Degallated species so retained on the column may be eluted using an appropriate solvent e.g. water, methanol, ethanol, acetone and mixtures thereof phase.

Alternatively, if anion exchange chromatography is used as a separation technique in step b), degallated species may be allowed to pass through a resin and gallic acid may be retained on to the column.

### Step c)

In step c), the tea material obtained at the end of step b) is subjected to drying.

Examples of drying techniques that may be used in step c) include freeze drying, spray drying, distillation, thermal evaporation and combinations thereof. Preferably, drying technique that may be used in step c) is thermal evaporation under vacuum. If thermal evaporation under vacuum is used in step c), the tea material obtained at the end of step b) is dried under vacuum in rotary evaporator at temperature preferably in the range below 50° C., more preferably below 40° C. to obtain DGTE.

The DGTE thus obtained comprises less than 0.2% gallic acid on dry weight basis and is used in the preparation of the composition. Preferably, the DGTE thus obtained further comprises from 5 to 25% caffeine on dry weight basis.

The composition comprises from 0.1 to 10 wt %, preferably from 0.25 to 8 wt %, more preferably from 0.5 to 7 wt %, even more preferably from 0.5 to 5 wt %, further more preferably from 0.5 to 3 wt %, yet more preferably from 0.5 to 2 wt % and most preferably from 0.5 to 1 wt % DGTE.

Preferably, the composition does not comprise any additional gallic acid other than that contained in the DGTE. Preferably, the composition does not comprise any additional caffeine than that contained in the DGTE.

### Additional Skin Lightening Agents

Preferably, the composition further comprises one or more skin lightening agents in addition to the DGTE.

Examples of skin lightening agents that may be used as an additional skin lightening agent in the composition include niacinamide, 12-hydroxystearic acid, resorcinol, phenylethyl resorcinol, 4-alkyl substituted resorcinol compounds, glutathione precursors, vitamin B6, vitamin C, vitamin A, galardin, adapalene, aloe extract, sage extract, ginger extract, ammonium lactate, arbutin, azelaic acid, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, glucopyranosyl-1-ascorbate, gluconic acid, glycolic acid, 4-Hydroxy-5-methyl-3[2H]-furanone, 4-hydroxyanisole and its derivatives, 4-hydroxybenzoic acid derivatives, hydroxycaprylic acid, inositol ascorbate, lactic acid, lemon extract, linoleic acid, magnesium ascorbyl phosphate, 5-octanoyl salicylic acid, salicylic acid, 3,4,5-trihydroxybenzyl derivatives, acetylglucosamine, pitera extract, symwhite, calcium pantothenate (Melano-block), seppiwhite, soybean extract (bowman birk inhibitor) and mixtures thereof. When 12-hydroxystearic acid is used in the composition, it is used as an additional skin lightening agent not as a fatty acid.

Preferably, skin lightening agents that may be used as an additional skin lightening agent in the composition are selected from niacinamide, 12-hydroxystearic acid, resorcinol, phenylethyl resorcinol, 4-alkyl substituted resorcinol compounds, glutathione precursors, and mixtures thereof. When used in the composition, 12-hydroxystearic acid is used as a skin lightening agent and not as a fatty acid.

Preferably, 4-alkyl substituted resorcinol compounds are selected from 4-methyl resorcinol, 4-ethyl resorcinol, 4-propyl resorcinol, 4-isopropyl resorcinol, 4-butyl resorcinol, 4-pentyl resorcinol, 4-hexyl resorcinol, 4-heptyl resorcinol, 4-octyl resorcinol and mixtures thereof. More preferably, 4-alkyl substituted resorcinol compounds selected are 4-ethyl resorcinol, 4-hexyl resorcinol and mixtures thereof.

When incorporated in the composition, an additional skin lightening agent may preferably be incorporated in an amount from 0.001 to 15 wt %, more preferably from 0.01 to 10 wt %, even more preferably 0.1 to 5 wt %, further more preferably form 0.5 to 3 wt %, in the composition.

### UVA Sunscreen

Preferably, the composition further comprises UVA organic sunscreens that absorbs UVA radiations and prevent them from reaching a surface e.g. skin of a user.

Examples of UVA organic sunscreens that may be used in the composition include dibenzoylmethane compound, bisdisulizole disodium (commercially available as Neo Heliopan® AP), diethylamino hydroxybenzoyl hexyl benzoate (commercially available as Uvinul® A Plus), Ecamsule (commercially available as Mexoryl SX) and Methyl anthranilate.

Preferably, UVA organic sunscreen that may be used as UVA sunscreen in the composition is selected from a dibenzoylmethane compound.

Examples of sunscreen of dibenzoymethane compound that may be used as UVA organic sunscreen in the composition include 4-tert-butyl-4′-methoxydibenzoylmethane (BMDM; commercially available as Parsol® 1789 or Avobenzone), 2-methyldibenzoylmethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane or 2,6-dimethyl-4-tert-butyl-4′-methoxy-dibenzoylmethane.

Most preferably, dibenzoylmethane compound that may be used as UVA organic sunscreen is BMDM.

When incorporated in the composition, UVA organic sunscreens may preferably be incorporated from 0.1 to 10 wt %, more preferably from 0.5 to 7 wt %, even more preferably from 1 to 5 wt %, further more preferably from 1 to 3.5 wt %, yet more preferably 1 to 3 wt %, still more preferably 1 to 2.5 wt % in the composition.

### UVB Organic Sunscreen

Preferably, the composition further comprises UVB organic sunscreens that absorbs UVB radiations and prevent them from reaching a surface e.g. skin of a user.

Examples of UVB organic sunscreens that may be used in the composition include compounds from the class of cinnamic acid, salicylic acid, diphenyl acrylic acid and derivatives thereof. Examples of such compounds include 2-ethylhexyl salicylate (commercially available as Octisalate™), 3,3,5-Trimethylcyclohexyl 2-hydroxybenzoate (commercially available as Homosalate™), Ethylhexyl Methoxycinnamate (commercially available as NeoHelipan® AV), 2-ethylhexyl 2-cyano-3,3-diphenylacrylate (OCR; commercially available as Octocrylene™), 2-Hydroxy-4-methoxybenzophenone (commercially available as Oxybenzone™), 2-ethyl-hexyl-4-methoxy cinnamate (MCX; commercially available as Parsol MCX™) and mixtures thereof. Preferably, UVB organic sunscreens that may be used in the composition are selected from OCR, MCX and mixtures thereof.

When incorporated in the composition, UVB organic sunscreens may preferably be incorporated from 0.1 to 10 wt %, more preferably from 0.5 to 7 wt %, even more preferably from 1 to 5 wt %, further more preferably from 1 to 3.5 wt %, yet more preferably from 1 to 3 wt %, still more preferably 1 to 2.5 wt % in the composition.

Preferably, the composition further comprises inorganic sunblock. Examples of inorganic sunblock that may be used in the composition include zinc oxide (ZnO), iron oxide, silica, such as fumed silica, or titanium dioxide (TiO₂). Preferably, inorganic sunblock that may be used in the composition are selected from TiO₂, ZnO and mixtures thereof.

When incorporated the composition, inorganic sunblock may preferably be incorporated from 0.1 to 10 wt %, more preferably from 0.5 to 7 wt %, even more preferably from 1 to 5 wt %, further more preferably from 1 to 3.5 wt %, yet more preferably from 1 to 3 wt %, still more preferably 1 to 2.5 wt %, in the composition.

### Fatty Acid

Preferably, the composition further comprises fatty acid. Fatty acids when present in a composition along with a soap provide the so called vanishing effect, i.e. a composition, when applied on to the human skin, vanishes on the skin leaving behind no significant streaks of the composition.

Preferably, fatty acids that may be present in the composition are selected from fatty acids that have 10 to 30, more preferably 12 to 25, even more preferably 14 to 20, further more preferably 16 to 18 carbon atoms.

Examples of fatty acids that may be used in the composition include pelargonic, lauric, myristic, palmitic, stearic, isostearic, oleic, linoleic, arachidic, behenic, erucic acid and mixtures thereof.

Preferably, fatty acid that may be present in the composition is selected from stearic acid, palmitic acid and mixtures thereof. The fatty acid in the present invention is preferably hystric acid which is substantially (generally about 90 to 95%) a mixture of stearic acid and palmitic acid in a ratio of between 55:45 to 45:55.

When incorporated, fatty acids may preferably be present from 4 to 25 wt %, more preferably from 6 to 22 wt %, even more preferably from 8 to 20 wt %, further more preferably from 10 to 19 wt % and yet more preferably from 12 to 18 wt %, in the composition.

### Soap

Preferably, the composition further comprises soap. Soap when present in combination with fatty acid in the composition provides vanishing effect.

Soap of the invention is generally prepared by in-situ neutralization of fatty acid present in the composition. Thus, it is preferred that the soap has a carbon chain length that corresponds to the chain length of fatty acid in the composition. The soap is formed from the fatty acid through use of alkali metal hydroxides e.g. sodium hydroxide or potassium hydroxide. Of the two, potassium hydroxide is more preferred. Thus, the soap is preferably a potassium soap (potassium salt of fatty acid).

Preferably, the composition comprises from 0.1 to 10 wt %, more preferably from 1 to 8 wt %, more preferably from 2 to 7 wt %, even more preferably from 3 to 6 wt % soap.

### Nonionic Surfactant

Preferably, the composition further comprises a nonionic surfactant having HLB value in the range 9 to 20, preferably 10 to 19, more preferably 12 to 18, even more preferably 13 to 17 and yet more preferably 15 to 17.

HLB is calculated using the Griffin method wherein HLB=20× Mh/M wherein Mh is the molecular mass of the hydrophilic portion of the molecule and M is the molecular mass of the whole molecule, giving a result on an arbitrary scale of 0 to 20. Typical values for various surfactants are given below:

A value <10: Lipid soluble (water insoluble)

A value >10: Water soluble

A value from 4 to 8 indicates an anti-foaming agent

A value from 7 to 11 indicates a W/O (water in oil) emulsifier

A value from 12 to 16 indicates oil in water emulsifier

A value from 11 to 14 indicates a wetting agent

A value from 12 to 15 is typical of detergents

A value of 16 to 20 indicates a solubiliser or a hydrotrope.

Preferably, the nonionic surfactant having HLB value in the range 9 to 20 is selected from fatty alcohol ethoxylates, alkyl phenol ethoxylates, polyoxyethylene sorbitan alkyl esters and mixtures thereof. Preferably, the nonionic surfactants are ones with at least 9 alkylene oxide groups preferably at least 9 ethylene oxide groups.

Examples of fatty alcohol ethoxylates that may be used as nonionic surfactants in the composition include polyoxyethylene lauryl ether (HLB=16.9; commercially available as Brij® 35), polyoxyethylene (20) cetyl ether (HLB=16; commercially available as Brij® 58), polyethylene glycol octadecyl ether (HLB=18.8; commercially available as Brij® 700) and Laureth—9 (C12EO9; HLB=14.3; commercially available as Brij® L9).

Examples of alkyl phenol ethoxylates that may be used as nonionic surfactant in the composition include octylphenol ethoxylate (HLB=15.5; commercially available as Triton™ X165), octylphenol ethoxylate (HLB=17.6; commercially available as Triton™ X405) and octylphenol ethoxylate (HLB=18.4; commercially available as Triton™ X705).

Examples of polyoxyethylene sorbitan alkyl esters that may be used as the nonionic surfactant in the composition include polyoxyethylenesorbitan monolaurate (HLB=13.3; commercially available as Tween® 21), polyoxyethylenesorbitan monolaurate (HLB=16.7; commercially available as Tween® 20), Polyoxyethylenesorbitan monopalmitate (HLB=15.6; commercially available as Tween® 40) and polyoxyethylene sorbitan monostearate (HLB=14.9; commercially available as Tween® 60).

More preferably, the nonionic surfactant having HLB value in the range 9 to 20 that may be present in the composition is fatty alcohol ethoxylate with saturated carbon chain having HLB higher than 15.5.

Preferably, the composition comprises from 0.5 to 5 wt %, more preferably from 1 to 4 wt %, even more preferably from 2 to 3 wt % nonionic surfactant having HLB in the range 9 to 20.

### Polymer

Preferably, the composition further comprises a polymer. The polymer acts as thickener in the composition and improves sensorial properties of the composition.

The polymer is preferably selected from the following classes:
acrylate/R-methacrylate copolymer e.g. acrylates/steareth-20 methacrylate copolymer (commercially available as Aculyn™ 22) and acrylates/beheneth-25 methacrylate copolymer (commercially available as Aculyn™ 28),
acrylate/R-methacrylate crosspolymer e.g. acrylates/steareth-20 methacrylate crosspolymer (commercially available as Aculyn™ 88),
acrylates copolymer (commercially available as Aculyn™ 33),
acrylate/R-alkyl acrylate crosspolymer e.g. acrylates/C10-C30 alkyl acrylate crosspolymer (commercially available as Pemulen™ TR-2),
copolymer of ammonium acryloyldimethyltaurate with vinyl pyrrolidone (commercially available as Aristoflex® AVC),
copolymer of sodium acryloyldimethyltaurate with vinyl pyrrolidone (commercially available as Aristoflex® AVS); and
crosspolymer of acryloyldimethyltaurate with R-alkyl acrylate and methyacrylate e.g. Ammonium acryloyldimethyltaurate/beheneth-25 methacrylate crosspolymer (commercially available as Aristoflex® HMB and Aristoflex® BLV).

Preferably, the composition comprises from 0.1 to 5 wt %, more preferably from 0.5 to 4.5 wt %, even more preferably from 1 to 4 wt %, further more preferably from 1.5 to 3.5 wt %, still more preferably from 2 to 3 wt % polymer.

Preferably, the composition comprises a cosmetically acceptable vehicle that includes water, and may be present in the composition in amount from 5 to 99.9 wt %, preferably from 10 to 95 wt %, more preferably from 15 to 90 wt %, even more preferably from 20 to 80 wt %, further more preferably from 25 to 75 wt % and still more preferably from 30 to 70 wt %.

Preferably, the composition further comprises emollients. Examples of emollients that may be used in the composition include stearyl alcohol, glyceryl monoricinoleate, mink oil, cetyl alcohol, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

Preferably, the composition further comprises solvents. Examples of solvents that may be used in the composition include ethyl alcohol, isopropanol, acetone, ethylene glycol monoethyl ether, diethylene glycol monobutyl ether, diethylene glycol monoethyl ether and mixtures thereof.

Preferably, the composition further comprises powders. Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Preferably, the composition further comprises preservatives to protect against the growth of potentially harmful microorganisms. Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. The alkane diols that are suitable for use as preservative are C₆-C₁₂ alkanes that are vicinally substituted with hydroxy groups. Illustrative examples include 1,2-octane diol (caprylyl glycol), 2,3-octane diol, 1,2-nonane diol, 1,2-decane diol, 1,2-hexane diol, 3,4-octane diol, mixtures thereof or the like where caprylyl glycol is typically the most preferred.

When present in the composition, preservatives are added preferably in an amount 0.001 to 5 wt %, more preferably 0.01 to 3 wt % and most preferably 0.02 to 2 wt %.

The composition may further comprise a range of other optional ingredients that include antioxidants, binders, biological additives, buffering agents, colorants, astringents, fragrance, opacifying agents, conditioners, exfoliating agents, pH adjusters, natural extracts, skin sensates, skin soothing agents, and skin healing agents.

The composition is preferably formulated in the form of a powder, flake, lotion, cream, gel or mousse. More preferably, the composition is formulated in the form of cream or lotion and most preferably in the form of cream. The composition can be a leave-on or wash-off type of composition. The composition is preferably a leave-on type of composition. The packaging for the composition of this invention can be a patch, bottle, tube, roll-ball applicator, propellant driven aerosol device, squeeze container or lidded jar.

In a second aspect, the present invention further relates to a method of providing skin lightening wherein the method comprises the steps of:
i. applying on the skin the composition according to the first aspect; and
ii. optionally, rinsing with water.

The present invention provides a method of providing skin lightening comprising the steps of applying the composition of the first aspect on to a surface e.g. skin, in case of a leave-on composition. This method optionally comprises an additional step of at least partially removing the composition from the surface if it is in the form of a wash-off composition. Preferably, the method is non-therapeutic. When the composition according to the first aspect is used according to the method of the second aspect, improved skin lightening is obtained.

In a third aspect, the present invention relates to use of the composition according to the first aspect for skin lightening. When the composition according to the first aspect is applied on to a surface e.g. skin, it provides improved skin lightening. Preferably, the use is non-therapeutic.

The present invention now will be demonstrated by way of following non-limiting examples.

### EXAMPLES

Materials:
Green tea (Synthite catalogue number SGT1006),
Black tea prepared using traditional black tea process using leaves of Camellia sinensis,
Glacial acetic acid (Merck-45754),
Polystyrene divinylbenzene resin (Diaion HP20 Supelco 13607),
Tannase (Kikkoman KT50)
Primary neonatal epidermal human melanocytes (Lonza catalogue number CC-2504),
EpiLife® Media (Cascade Biologics catalogue number MEPI500CA),
Dulbecco's modified Eagle's Medium (DMEM; Gibco catalogue number 31600034),
MBM-4 Melanocyte Growth Basal Medium (Lonza catalogue number CC-3250); and
α-Melanocyte stimulating hormone (α-MSH; R&D System-3013).

### Preparation of the DGTE

### Step a): Subjecting a Tea Material to an enzymatic hydrolysis

A 10% solution of black tea was prepared in de-ionized water. If required, this solution was heated up to 70° C. to dissolve black tea in water. To this, 10 mL of tannase solution (5 mg/mL) was added and the enzymatic hydrolysis was carried out for 60 minutes at 40° C.

Such enzyme hydrolyzed tea material was either subjected to step b) or stored in an amber colored container and is stored at 4° C. until used in step b).

### Step b): Separating Gallic Acid from the Enzyme Hydrolyzed Tea Material

Tea material obtained after step a) was subjected to adsorption chromatography to separate gallic acid from the enzyme hydrolyzed tea material obtained at the end of step a).

The tea material obtained at the end of step a) was mixed with 2 mL glacial acetic acid and this acidified mixture was loaded on to a column containing polystyrene divinylbenzene resin. The resin used has high affinity for flavonoids e.g. EGC and EC, whereas the resin has poor affinity towards phenolic acids e.g. gallic acid. The column was eluted with 2% acetic acid (aqueous). The elute was analyzed for gallic acid content using HPLC. The elution step with 2% acetic acid was carried out until gallic acid content as analyzed by HPLC was found to be (10 mM). After this, the column was eluted with 50% aqueous ethanol.

### Step c): Drying the Tea Material Obtained in Step b)

Tea material obtained after step b) was subjected to drying under vacuum while maintain temperature <50° C. The DGTE obtained after drying was stored in an air tight glass container.

### Cell Culture

Human primary melanocytes were grown in melanocytes growth medium (MGM) with human melanocyte growth supplement. 500 μL/well (5×10⁴ cells/well) of this solution was plated in a 24 wells plate and incubated in an incubator (Thermo Scientific, Model 3111) at 37° C. with 5% CO₂ atmosphere.

### Melanin Content Assay

After 24 h of incubation, cell cultures were treated with the test actives (as outlined in table 1) dissolved in 20% ethanol. After 1 h of actives addition, 10 nM of α-MSH was added. The cells were again incubated for 72 h in an incubator. At the end of this incubation period, the culture medium was replaced with 120 μL/well of melanin content assay reagent (10% Dimethyl sulfoxide in 1 N sodium hydroxide) and incubated for 1 h at 60° C. in a shaker incubator. The supernatant was then transferred to a 384 well plate. The absorbance was measured at 405 nm with a microtiter plate reader (Genios Pro, Tecan M 1000).

### Cell Viability Assay

Viability of cells was assessed using Calcein-AM viability assay. Briefly, spent media was removed from all wells and cells were rinsed once with 400 μL of phosphate buffered saline including wells without cells (control wells). To all these wells, 1 μM Calcein-AM (working stock) prepared in PBS was added (200 μL (in case 23 well plate was used) or 100 μL (in case a 96 well plate is used)). The plate was covered with aluminium foil completely and incubated for 30 min in CO₂ incubator. Post this, fluorescence (excitation at 490 nm and emission at 520 nm) was measured with a microtiter bottom plate reader (Genios Pro, Tecan M 1000).

In the first set of experiments, human primary melanocytes were subjected to treatments as outlined in table 1 below.

TABLE 1

| Example | MSH | 4-ER | Black Tea | DGTE^{#} |
|---|---|---|---|---|
| A | — | — | — | — |
| (Control; no treatment) | | | | |
| B | 10 nM | — | — | — |
| C | 10 nM | 50 μM | — | — |
| D | 10 nM | — | 25 ppm | — |
| 1 | 10 nM | — | — | 25 ppm |

^{#}obtained using black tea of example D in step a) of preparation of the DGTE.

After incubation, melanin content of melanocytes subjected to treatment outlined in table 1 was estimated as described earlier and was found to be as in the table 2 below.

TABLE 2

| | Melanin Content (%) |
|---|---|
| Example | Average ± SD |
| A | 100 |
| B | 121 ± 9.7 |
| C | 68 ± 12.7 |
| D | 106 ± 7 |
| 1 | 89 ± 7.9 |

The data in table 1 above shows that melanin content of example B, where cells were treated with MSH; a known inducer of melanin formation, was found to be over and above the control cells (example A; no treatment). As described in experimental section, melanocytes used in all the examples in table 1 were treated with the same amount of MSH (10 nM). Despite MSH being present, melanin content of the melanocytes treated with the DGTE (example 1) was found to have reduced as compared to melanin content of cells treated black tea (example D). This indicates that the DGTE which comprises less than 0.2% gallic acid on dry weight basis (Example 1) provided improved skin lightening in such MSH induced condition as compared to skin lightening provided by control (example D).

In conclusion, a topical composition comprising a degallated tea extract wherein the extract comprises less than 0.2% gallic acid on dry weight basis, provides improved skin lightening.

1. A topical composition comprising from 0.01 to 10 wt % degallated tea extract wherein the extract comprises less than 0.2% gallic acid on dry weight basis.

2. The composition according to claim 1 wherein the extract further comprises from 5 to 25% caffeine on dry weight basis.

3. The composition according to claim 1 wherein the extract is obtained using tea material obtained from Camellia sinensis.

4. The composition according to claim 1 wherein the composition further comprises from 0.001 to 15 wt % skin lightening agent selected from niacinamide, 12-hydroxystearic acid, resorcinol, phenylethyl resorcinol, 4-alkyl substituted resorcinol compounds, glutathione precursors and mixtures thereof.

5. The composition according to claim 1 wherein the composition further comprises from 0.1 to 10 wt % UVA organic sunscreen.

6. The composition according to claim 1 wherein the composition further comprises from 0.1 to 10 wt % UVB organic sunscreen.

7. The composition according to claim 1 wherein the composition further comprises from 4 to 25 wt % fatty acid.

8. The composition according to claim 1 wherein the composition further comprises from 0.1 to 10 wt % soap.

9. The composition according to claim 1 wherein the composition further comprises a nonionic surfactant having an HLB value in the range 9 to 20.

10. The composition according to claim 1 wherein the composition further comprises a polymer.

11. The composition according to wherein the composition is a leave-on composition.

12. A cosmetic method of providing skin lightening wherein the method comprises the steps of:
i. applying on the skin the composition according to claim 1; and
ii. optionally, rinsing with water.

13. Cosmetic use of the composition according to claim 1 for skin lightening.

14. A topical composition for use as a medicament to provide skin lightening, comprising from 0.01 to 10 wt % degallated tea extract wherein the extract comprises less than 0.2% gallic acid on dry weight basis.
